(19) United States
(12) Patent Application Publication
(10) Pub. No.: US 2021/0318254 A1
(43) Pub. Date: Oct. 14, 2021

(54) GRID AND GRID-EQUIPPED X-RAY DETECTOR

(71) Applicant: MITAYA MANUFACTURING CO., LTD., Kanazawa-shi, Saitama (JP)

(72) Inventor: Katsuhiro ICHIKAWA, Kanazawa-shi, lshikawa (JP)

(21) Appl. No.: 17/277,273

(22) PCT Filed: Sep. 6, 2019
(86) PCT No.: PCT/JP2019/035214

§ 371 Date: Mar. 17, 2021

(30) Foreign Application Priority Data
Sep. 19, 2018 (JP) 2018-174609

(51) Int. Cl. G01N 23/04; H04N 5/32

(52) U.S. Cl. CPC G01N 23/04; G01N 2223/04; H04N 5/32

(57) ABSTRACT

A grid of the present invention is used with an X-ray detector to take an X-ray image of a subject. The grid is configured by alternately arranging a plurality of X-ray absorption parts and a plurality of X-ray transmission parts and adapted to be externally provided adjacent to a surface of the X-ray detector. When the grid is viewed in a plan view in a state that the grid is provided adjacent to the surface of the X-ray detector, at least at least a part of the X-ray absorption part overlaps with an imaging part of the X-ray detector and an overlapping state of the X-ray absorption part and the imaging part transitions. Further, the grid includes the plurality of X-ray absorption parts having the same overlapping state with the corresponding imaging parts in a predetermined cycle.

### TECHNICAL FIELD

The present invention relates to a grid adapted to be externally provided on a surface of an X-ray detector and used to remove scattered X-rays scattered by a subject in an X-ray imaging apparatus, and a grid-equipped X-ray detector.

### RELATED ART

In recent years, as an X-ray imaging apparatus used in X-ray diagnosis, a digital X-ray imaging apparatus using a digital X-ray detector such as CR (Computed Radiography) or FPD (Flat Panel Detector) has been used.

In the X-ray imaging apparatus, a grid for removing scattered X-rays scattered by a subject is provided on the subject side of the X-ray detector. Such a grid has a configuration in which X-ray absorption parts made of a material absorbing X-rays and X-ray transmission parts made of a material transmitting X-rays are alternately arranged. On the other hand, the X-ray detector has a configuration in which pixels each including an imaging part for detecting incident X-rays and a non-imaging part provided adjacent to the imaging part are two-dimensionally arranged.

In the X-ray imaging apparatus including the grid and the X-ray detector, a pitch (arrangement interval) of the X-ray absorption parts of the grid and a pitch of the pixels of the X-ray detector are generally different. Due to this difference, in an X-ray image obtained by the X-ray detector, grid artifacts caused by a transition of a position of the X-ray absorption part for each pixel occur. When such grid artifacts occur in the X-ray image, an image quality of the X-ray image deteriorates so that diagnosis and treatment of a disease cannot be performed properly. Therefore, measures have been taken to eliminate or reduce the grid artifacts occurring in the X-ray images.

Specifically, there is known the X-ray imaging apparatus using a structure in which the X-ray absorption parts are arranged at the same pitch as the pixels of the X-ray detector and the X-ray detector and the grid are integrated so that the non-imaging part of the pixel of the X-ray detector and the X-ray absorption part are aligned in a plan view (for example, Patent Document 1). In this X-ray imaging apparatus, it is theoretically possible to prevent occurrence of the grid artifacts. Such a structure is achieved by arranging X-ray absorption parts and X-ray transmission parts on a surface of the X-ray detector in such a manner that the pitch of the pixels matches the pitch of the X-ray absorption parts and integrally assembling them.

However, when the grid adapted to be externally attached to the X-ray detector is used, it is difficult to match the pitch of the X-ray absorption parts of the grid with the pitch of the pixels of the X-ray detector as in the above structure. The reason for this is that such a grid is manufactured by alternately bonding the X-ray absorption parts and the X-ray transmission parts with an adhesive or the like, so that the pitch of the X-ray absorption parts cannot be set exactly. In addition, the grid needs to be accurately aligned with the X-ray detector. Such a grid is difficult to manufacture practically, and as a result, deterioration of the quality of the X-ray image due to the influence of the grid artifacts cannot be sufficiently suppressed.

### PRIOR ART DOCUMENT

### Patent Document

Patent document 1: JP-A-H9-75332

### SUMMARY OF THE INVENTION

### Problem to be Solved by the Invention

It is an object of the present invention to provide a grid for removing scattered X-rays, which is adapted to be externally attached to an X-ray detector and enables to perform accurate X-ray imaging of a subject while preventing deterioration of the quality of X-ray images due to grid artifacts. Further, it is another object of the present invention to provide a grid-equipped X-ray detector having the grid.

### Means for Solving the Problem

Such an object is achieved by the present inventions (1) to (7) described below.

(1) A grid used to be with an X-ray detector to take an X-ray image of a subject, wherein the X-ray detector is configured by two-dimensionally arranging a plurality of pixels each including an imaging part for detecting incident X-rays and a non-imaging part provided adjacent to the imaging part, the grid comprising:

a plurality of X-ray absorption parts that absorb the X-rays; and

a plurality of X-ray transmission parts that transmit the X-rays,

wherein the grid is configured by alternately arranging the plurality of X-ray absorption parts and the plurality of X-ray transmission parts and adapted to be externally provided adjacent to a surface of the X-ray detector,

wherein when the grid is viewed in a plan view in a state that the grid is provided adjacent to the surface of the X-ray detector, at least a part of the X-ray absorption part overlaps with the imaging part and an overlapping state of the X-ray absorption part and the imaging part transitions,

wherein the plurality of X-ray absorption parts include first X-ray absorption parts having one end which overlaps with one end of the non-imaging parts, and

wherein the first X-ray absorption parts are included in the grid at a predetermined cycle and the predetermined cycle is 7.5 mm or more and 310 mm or less.

(2) The grid described in the above-mentioned item (1), wherein the X-ray absorption part further overlaps with the non-imaging part.

(3) The grid described in the above-mentioned item (1) or (2), wherein when a width at which the at least part of the X-ray absorption part overlaps with the imaging part is defined as X [μm] and a width of the imaging part is defined as Y [μm], a value of X/Y is in the range of 0.01 to 0.30.

(4) The grid described in the above-mentioned items (1) to (3), wherein at least a part of the X-ray transmission part overlaps with the non-imaging part.

(5) The grid described in the above-mentioned items (1) to (4), wherein a pitch of the plurality of X-ray absorption parts is smaller than a pitch of the plurality of pixels.

(6) The grid described in the above-mentioned items (1) to (5), wherein the X-ray transmission part is made of a paper fibrous base material impregnated with an epoxy resin.

(7) A grid-equipped X-ray detector comprising:

an X-ray detector configured by two-dimensionally arranging a plurality of pixels each including an imaging part for detecting incident X-rays and a non-imaging part provided adjacent to the imaging part; and

a grid having a plurality of X-ray absorption parts that absorb the X-rays and a plurality of X-ray transmission parts that transmit the X-rays, the grid configured by alternately arranging the plurality of X-ray absorption parts and the plurality of X-ray transmission parts and adapted to be externally provided adjacent to a surface of the X-ray detector,

wherein when the grid is viewed in a plan view in a state that the grid is provided adjacent to the surface of the X-ray detector, at least a part of the X-ray absorption part overlaps with the imaging part of the X-ray detector and an overlapping state of the X-ray absorption part and the imaging part transitions, and

wherein the plurality of X-ray absorption parts are included in the grid at a predetermined cycle and the predetermined cycle is 7.5 mm or more and 310 mm or less.

### Effects Of The Invention

According to the present invention, when the grid is viewed in a plan view in a state that the grid is provided adjacent to the surface of the X-ray detector, at least a part of the X-ray absorption part of the grid overlaps with the imaging part of the X-ray detector and an overlapping state of the X-ray absorption part and the imaging part transitions. Further, as a result of the transition of this overlapping state, the grid includes the plurality of X-ray absorption parts (the first X-ray absorption parts) having the same overlapping state with the corresponding imaging parts in a predetermined cycle, and the predetermined cycle is set within the range of 7.5 mm or more and 310 mm or less.

In the present invention, reduction of brightness in the X-ray image due to the X-ray absorption part of the grid also transitions with the transition of the overlapping state of the X-ray absorption parts of the grid and the imaging parts, thereby grid artifacts (grid lines) that form periodic lines occur. However, since the plurality of first X-ray absorption parts having the same overlapping state with the corresponding imaging parts are included in the grid at a predetermined cycle (7.5 mm or more and 310 mm or less), a pitch of the grid lines becomes longer and a frequency thereof becomes lower. Further, as the pitch of the grid lines becomes longer, its amplitude becomes smaller. Therefore, the grid lines in the X-ray image can be made inconspicuous. In addition, the cycle of the first X-ray absorption parts included in the grid matches the pitch of the grid lines. Further, in the case where an arrangement direction of the X-ray absorption parts of the grid and an arrangement direction of the pixels of the X-ray detector are not completely parallel, the grid lines become a set of diagonal lines to hinder the diagnosis, but the effect of the longer pitch of the grid lines can make the grid lines inconspicuous as well.

For these reasons, by using the grid of the present invention, it is possible to obtain a clear high-quality X-ray image in which the grid lines and the grid artifacts are not visually recognized without exactly matching the pitch of the X-ray absorption parts of the grid with the pitch of the pixels of the X-ray detector and accurately aligning the grid with respect to the X-ray detector. As a result, this makes it possible to perform accurate X-ray imaging of a subject.

### BRIEF DESCRIPTION OF THE DRAWINGS

FIG. 1 is a cross-sectional view showing a state where a preferred embodiment of a grid of the present invention is attached to an X-ray detector (a preferred embodiment of a grid-equipped X-ray detector of the present invention).

FIG. 2(a) is a partially enlarged view of a region (a) of the grid-equipped X-ray detector shown in FIG. 1, and FIG. 2(b) is a partially enlarged view of a region (b) of the grid-equipped X-ray detector shown in FIG. 1.

FIGS. 3(a) to (c) are schematic diagrams showing a relationship between a pitch and an amplitude of grid lines due to a difference between a pitch of the X-ray absorption part of the grid and a pitch of the pixels of the X-ray detector.

FIG. 4 is a graph showing an example of a relationship between a spatial frequency of the grid lines and a contrast sensitivity of an observer.

FIG. 5 is a cross-sectional view showing a state where a grid of another preferred embodiment of the present invention is attached to an X-ray detector.

FIGS. 6(a) and (b) are diagrams showing the grid lines appearing in X-ray images obtained by directly irradiating the grid-equipped X-ray detectors of Examples 1 and 2 with X-rays.

FIG. 7 is a diagram showing X-ray images obtained by imaging a subject (a knee joint part of a human body) using the grid-equipped X-ray detectors of Example 3 and Comparative Example 1.

### MODE FOR CARRYING OUT THE INVENTION

Hereinafter, detailed description will be made on a grid and a grid-equipped X-ray detector of the present invention based on preferred embodiments described in the accompanying drawings.

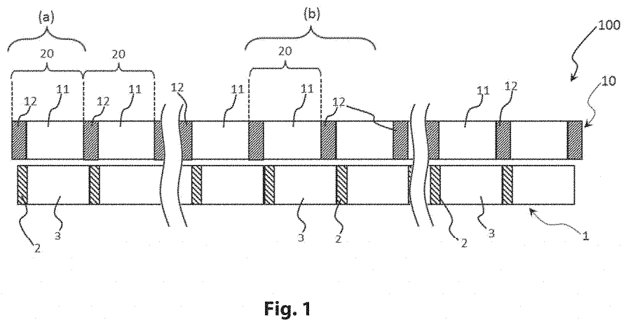

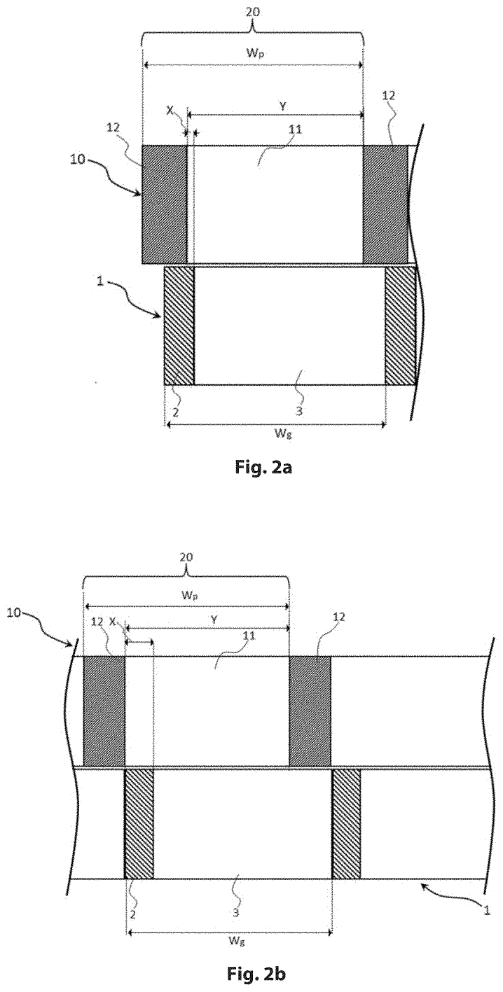

FIG. 1 is a cross-sectional view showing a state where a preferred embodiment of a grid of the present invention is attached to an X-ray detector (a preferred embodiment of a grid-equipped X-ray detector of the present invention). FIG. 2(a) is a partially enlarged view of a region (a) of the grid-equipped X-ray detector shown in FIG. 1, and FIG. 2(b) is a partially enlarged view of a region (b) of the grid-equipped X-ray detector shown in FIG. 1.

A grid of the present invention is adapted to be externally provided on a surface of an X-ray detector and is used in an X-ray imaging apparatus for taking an X-ray image of a subject.

### Grid-Equipped X-Ray Detector

A grid-equipped X-ray detector 100 shown in FIG. 1 has an X-ray detector 10 that detects X-rays passed through the subject and a grid 1 adapted to be externally attached to a surface of the X-ray detector 10 on the subject side for removing X-rays (scattered X-rays) scattered by the subject.

Prior to the description of the grid 1 in detail, the X-ray detector 10 to which the grid 1 is to be attached will be described. X-ray detector

The X-ray detector 10 has a plurality of pixels 20 each including an imaging part 11 for detecting incident X-rays and a non-imaging part 12 provided adjacent to the imaging part 11. The X-ray detector 10 shown in FIG. 1 is configured by two-dimensionally arranging the plurality of pixels 20. More specifically, the plurality of pixels 20 are arranged in a two-dimensional array to form the X-ray detector 10.

Examples of the X-ray detector 10 include digital X-ray detectors such as an indirect conversion type FPD (Flat Panel Detector) and a direct conversion type FPD.

In the indirect conversion type FPD, the pixel 20 is composed of an X-ray detection element formed of a phosphor such as thallium-activated cesium iodide (CsI:T1), a photodiode, a storage capacitor, a TFT switch, and the like. Although the specific configuration is not shown, in the indirect conversion type FPD, an intensity of X-rays (X-ray signal) incident on the imaging part 11 is converted into the intensity of light (optical signal) by the phosphor. This optical signal is converted into a charge signal by the photodiode, and further, a current signal is passed through the signal line by the TFT switch. Then, the current signal is input to an image processing device (computer) as a digital signal encoded through an A/D (Analogue Digital) converter, and a two-dimensional image (X-ray image) is formed.

Further, in the direct conversion type FPD, the X-ray detection element constituting the pixel 20 has a structure in which a voltage is applied to amorphous selenium (a-Se) as the phosphor, and the direct conversion type FPD is mainly used for mammography. The direct conversion type FPD forms the X-ray image in the same manner as the indirect conversion type FPD described above, except that the X-ray signal incident on the imaging part 11 is directly converted into a negative and positive charge signal by the a-Se to which the voltage is applied.

Further, the grid of the present invention can be used for both the direct conversion type FPD and the indirect conversion type FPD as the X-ray detector 10, but in particular, when used in combination with the indirect conversion type FPD, a clearer X-ray image can be obtained.

A width of the imaging part 11 is not particularly limited and is in the range of about 50 to 150 μm, but in the present embodiment, it is set to 120 μm. A width of the non-imaging part 12 is not particularly limited and is in the range of about 10 to 50 μm, but in the present embodiment, it is set to 30 μm. Therefore, a width of each pixel 20 (a pitch) of the X-ray detector 10 shown in FIG. 1 is 150 μm.

A size of the X-ray detector 10 in a plan view is not particularly limited, but is, for example, about 200 mm×200 mm.

### Grid

Next, the grid (externally provided grid) 1 of the present embodiment will be described.

The grid 1 includes a plurality of X-ray absorption parts 2 that absorb X-rays and a plurality of X-ray transmission parts 3 that transmit X-rays. More specifically, the grid 1 shown in FIG. 1 is configured by alternately arranging plate-shaped X-ray absorption parts 2 and plate-shaped X-ray transmission parts 3.

A constituent material of the X-ray absorption part 2 is not particularly limited as long as it is a material that absorbs X-rays, but, for example, lead can be used.

In the present embodiment, when the grid 1 is viewed in a plan view in a state that the grid 1 is provided adjacent to the surface of the X-ray detector 10, the grid 1 is attached to the X-ray detector 10 in such a manner that at least a part of the X-ray absorption part 2 overlaps with the imaging part 11 (see FIG. 2).

A thickness of the X-ray absorption part 2 is appropriately changed depending on a thickness (the width) of the non-imaging part 12 of the X-ray detector 10, but specifically, is preferably in the range of about 15 to 40 μm, and more preferably in the range of about 18 to 30 μm. In the case where the thickness of the X-ray absorption part 2 is within the above range, when the grid 1 is viewed in the plan view, the part of the X-ray absorption part 2 overlaps with the imaging part 11 and an area of the overlapping region can be made sufficiently small. As a result, the X-rays passed through the subject are made incident on the imaging part 11 more efficiently so that more accurate X-ray imaging of the subject can be performed.

A constituent material of the X-ray transmission part 3 is not particularly limited as long as it transmits X-rays or has an extremely low X-ray absorption rate, and examples thereof include aluminum, a base material made of a synthetic resin, a fibrous base material, a fibrous base material impregnated with a synthetic resin, and the like. Examples of the synthetic resins include various kinds of thermoplastic resins such as polyolefin (e.g., polyethylene or polypropylene), polyamide, polyester, polyphenylene sulfide, polycarbonate, polymethyl methacrylate and polyether; various kinds of thermosetting resins such as epoxy resin and acrylic resin; various kinds of thermoplastic elastomers; and the like. Further, examples of the fibrous base material include a paper fibrous base material, a carbon fibrous base material, a glass fibrous base material and the like.

Among them, by using the paper fibrous base material impregnated with an epoxy resin (paper epoxy) as the constituent material of the X-ray transmission part 3, an X-ray transmittance of the X-ray transmission part 3 can be increased. Further, the paper fibrous base material impregnated with the epoxy resin has advantages in that it can be easily processed as compared to another base material and can be manufactured at a low cost.

Further, the X-ray absorption parts 2 and the X-ray transmission parts 3 are adhered to each other by an epoxy adhesive or the like. Since the X-ray absorption rate of such an adhesive is extremely low like the X-ray transmission part 3, it can be regarded as a part of the X-ray transmission part 3 in the grid 1.

A thickness of the X-ray transmission part 3 is appropriately changed depending on the width (the pitch) of the pixel 20, but specifically, is preferably in the range of about 110 to 190 μm, and more preferably in the range of about 125 to 180 μm.

A size of the grid 1 in the plan view is not particularly limited, but it is preferred that the size is substantially the same size as the X-ray detector 10, and more preferably about 200 mm×200 mm.

In the present embodiment, when the grid 1 is viewed in the plan view in a state that the grid 1 is provided adjacent to the surface of the X-ray detector 10, the grid 1 is configured in such a manner that at least a part of the X-ray absorption part 2 overlaps with the imaging part 11 of the X-ray detector 10. Therefore, in the grid-equipped X-ray detector 100 shown in FIG. 1, at least a part of the X-ray absorption part 2 overlaps with the imaging part 11 of each pixel 20. In this state, an overlap is minimized (or none) when one end of the X-ray absorption parts 2 overlaps with one end of the non-imaging parts 12, and an overlapping state transitions according to a change of a position of the X-ray absorption part 2. By the transition of this overlapping state, the grid 1 includes a plurality of X-ray absorption parts 2 having the same overlapping state as the corresponding imaging parts 11 at a predetermined cycle, and the predetermined cycle is 7.5 mm or more and 310 mm or less.

In the present embodiment, reduction of brightness in the X-ray image due to the X-ray absorption parts 2 of the grid 1 also transitions with the transition of the overlapping state of the X-ray absorption parts 2 of the grid 1 and the imaging parts 11, and grid artifacts (grid lines) that form periodic lines occur. However, since the plurality of X-ray absorption parts 2 having the same overlapping state as the imaging parts 11 are included in the grid 1 at the predetermined cycle (7.5 mm or more and 310 mm or less), a pitch of the grid lines becomes longer and a frequency thereof becomes lower. Therefore, the grid lines in the X-ray image can be made inconspicuous. In addition, the cycle of the X-ray absorption parts 2 having the same overlapping state as the imaging parts 11 included in the grid 1 matches the pitch of the grid lines.

Further, in the case where an arrangement direction of the X-ray absorption parts 2 of the grid 1 and an arrangement direction of the pixels 20 of the X-ray detector 10 are not completely parallel, the grid lines become a set of diagonal lines to hinder the diagnosis, but the effect of the longer pitch of the grid lines can make the grid lines inconspicuous as well.

Therefore, by using the grid 1, it is possible to obtain a clear high-quality X-ray image in which the grid lines and the grid artifacts are not visually recognized without exactly matching the pitch of the X-ray absorption parts 2 of the grid 1 with the pitch of the pixels 20 of the X-ray detector 10 and accurately aligning the grid 1 with respect to the X-ray detector 10. As a result, this makes it possible to perform accurate X-ray imaging of a subject.

In the present embodiment, the predetermined cycle is 7.5 mm or more and 310 mm or less, but is preferably 22.5 mm or more and 310 mm or less, and more preferably 45 mm or more and 310 mm or less. When the predetermined cycle is within the above range, the pitch of the grid lines appearing in the X-ray image becomes longer and the frequency thereof becomes lower. Therefore, the grid lines in the X-ray image can be made more inconspicuous.

Further, in the present embodiment, it is preferable to set a difference between the total width of the X-ray absorption part 2 and the X-ray transmission part 3 (the pitch of the X-ray absorption parts 2) of the grid 1 and the width of the pixel 20 (the pitch of the pixel 20) of the X-ray detector 10 within a predetermined range. Specifically, when the total width of the X-ray absorption part 2 and the X-ray transmission part 3 of the grid 1 is defined as W_{g} [μm] and the width of the pixel 20 of the X-ray detector 10 is defined as Wₚ [μm], it is preferable to configure the grid 1 so as to satisfy a relationship of −3.0≤W_{g}−Wₚ≤3.0 (however, W_{g}−Wₚ=0 is excluded). As a result, the pitch of the grid artifacts (grid lines) appearing in the X-ray image due to the difference between the pitch (W_{g}) of the X-ray absorption parts 2 of the grid 1 and the pitch (Wₚ) of the pixels 20 of the X-ray detector 10 can be made long.

Further, it is preferred that the grid 1 satisfies the relationship of −1.0≤W_{g}−Wₚ≤1.0 (however, W_{g}−Wₚ=0 is excluded), and it is more preferred that the grid 1 satisfies the relationship of −0.5≤W_{g}−Wₚ≤0.5 (however, W_{g}−Wₚ=0 is excluded). As a result, the pitch of the grid artifacts (grid lines) appearing in the X-ray image can be made longer.

Here, a relationship between the difference between the pitch (W_{g}) of the X-ray absorption parts of the grid and the pitch (Wₚ) of the pixels of the X-ray detector, and the grid lines appearing in the X-ray image will be described.

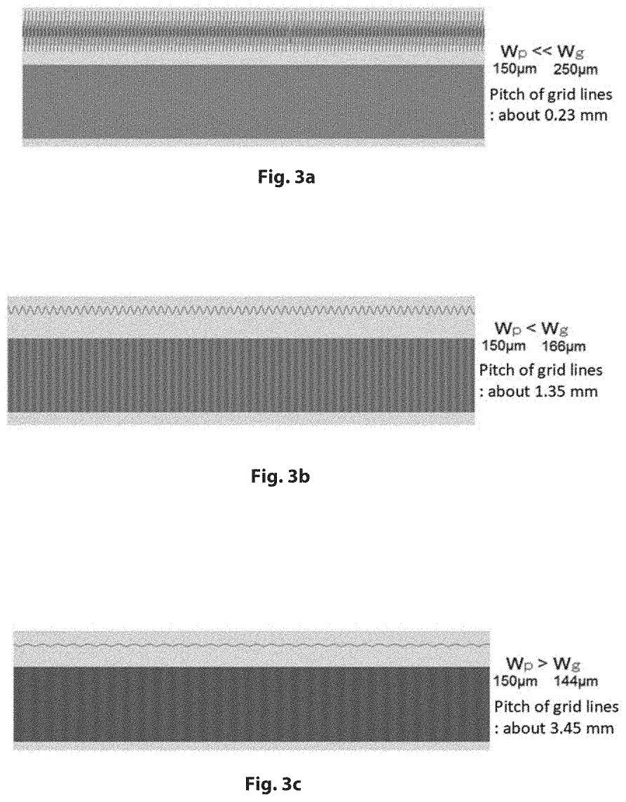

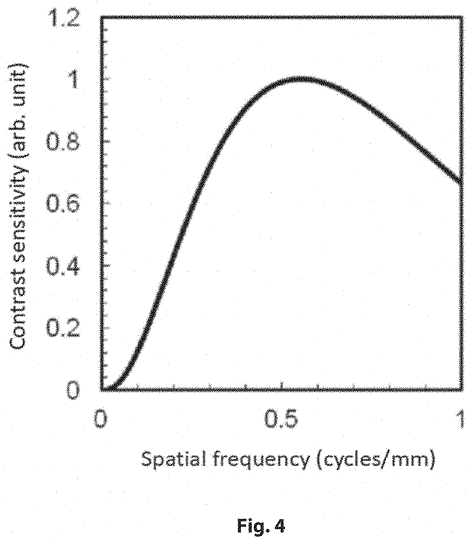

FIGS. 3(a) to (c) are schematic diagrams showing a relationship between a pitch and an amplitude of grid lines due to a difference between a pitch of the X-ray absorption parts of the grid and a pitch of the pixels of the X-ray detector. FIG. 4 is a graph showing an example of a relationship between a spatial frequency of the grid lines and a contrast sensitivity of an observer. In the following description, the pitch (Wₚ) of the pixels of the X-ray detector is 150 μm.

FIG. 3(a) is the diagram showing the pitch and the amplitude of the grid lines appearing in the X-ray image when the grid-equipped X-ray detector in which the grid with the pitch (W_{g}) of the X-ray absorption parts being 250 μm is provided adjacent to the surface on the subject side (X-ray light source side) of the X-ray detector is irradiated with the X-rays. Therefore, the value of W_{g}−Wₚ is 100 μm.

FIG. 3(b) is the diagram showing the pitch and the amplitude of the grid lines appearing in the X-ray image when the grid-equipped X-ray detector in which the grid with the pitch (W_{g}) of the X-ray absorption parts being 166 μm is provided adjacent to the surface on the subject side (X-ray light source side) of the X-ray detector is irradiated with the X-rays. Therefore, the value of W_{g}−Wₚ is 16 μm.

FIG. 3(c) is the diagram showing the pitch and the amplitude of the grid lines appearing in the X-ray image when the grid-equipped X-ray detector in which the grid with the pitch (W_{g}) of the X-ray absorption parts being 144 μm is provided adjacent to the surface on the subject side (X-ray light source side) of the X-ray detector is irradiated with the X-rays. Therefore, the value of W_{g}−Wₚ is −6 μm.

As shown in FIGS. 3(a) to 3(c), as the difference between the pitch (W_{g}) of the X-ray absorption parts and the pitch (Wₚ) of the pixels becomes smaller, the pitch of the grid lines appearing in the X-ray image becomes longer and the frequency thereof becomes lower. Specifically, the grid lines appearing in FIG. 3(a) have the pitch of about 0.23 mm and the frequency (spatial frequency) of about 4.41 (cycles/mm), and similarly, the grid lines appearing in FIG. 3(b) have the pitch of about 1.35 mm and the frequency of about 0.74 (cycles/mm), and the grid lines appearing in FIG. 3(c) have the pitch of about 3.45 mm and the frequency of about 0.29 (cycles/mm).

As described above, the pitch of the grid lines appearing in the X-ray image matches the cycle of X-ray absorption parts 2 having the same overlapping state with the imaging parts 11 included in the grid 1. Therefore, as the difference between the pitch (W_{g}) of the X-ray absorption parts and the pitch (Wₚ) of the pixels becomes smaller, the cycle of the X-ray absorption parts 2 having the same overlapping state with the imaging parts 11 becomes longer.

As the pitch of the grid lines becomes longer, its amplitude becomes smaller. Further, as the pitch of the grid lines becomes longer, an area occupied by the grid lines with respect to the entire X-ray image decreases. Therefore, by reducing the difference between the pitch (W_{g}) of the X-ray absorption parts and the pitch (Wₚ) of the pixels, an influence of the grid lines appearing in the X-ray image can be reduced.

Further, as shown in FIG. 4, when the spatial frequency of the grid lines becomes smaller than about 0.5 (cycles/mm), human visual response (contrast sensitivity) decreases. The grid-equipped X-ray detector 100 of the present embodiment is configured so that the difference between the pitch (W_{g}) of the X-ray absorption parts and the pitch (Wₚ) of the pixels is 3 μm or less, so that the grid lines appearing in the X-ray image have extremely low frequency. Specifically, the frequency (spatial frequency) of the grid lines is about 0.13 (cycles/mm) or less, and the pitch of the grid lines is about 7.5 mm or more. Therefore, when the difference between the pitch (W_{g}) of the X-ray absorption parts and the pitch (Wₚ) of the pixels is 3 μm or less, a cycle in which the plurality of X-ray absorption parts 2 having the same overlapping state with the imaging parts 11 are included in the grid 1 is within the above-mentioned predetermined range. When the frequency of the grid lines is about 0.13 (cycles/mm) or less, the difference in contrast between the grid lines in the X-ray image and the periphery of the grid lines cannot be recognized.

Therefore, by setting the difference between the pitch (W_{g}) of the X-ray absorption parts and the pitch (Wₚ) of the pixels to 3 μm or less, the grid lines appearing in the X-ray image become inconspicuous.

Further, in the grid-equipped X-ray detector 100 of the present embodiment, the grid lines in the obtained X-ray image are inconspicuous. Therefore, it is not necessary to perform image processing, which has been conventionally performed, for eliminating image signals of the grid lines from the X-ray image.

In the grid-equipped X-ray detector 100 of the present embodiment, at least a part of the X-ray absorption part 2 overlaps with the imaging part 11 of the X-ray detector 10 in the plan view of the grid 1. Therefore, due to the above-mentioned action, the grid lines have a long pitch and a low frequency, and the grid line becomes inconspicuous. On the other hand, when the difference between W_{g} and Wₚ becomes large (for example, the value of W_{g}−Wₚ is 5 μm or more), the pitch of the grid lines becomes small and its amplitude increases. Therefore, the influence of the grid artifacts is remarkably exhibited, and it becomes impossible to obtain a high-quality X-ray image.

In the grid-equipped X-ray detector 100 of the present embodiment, when a width of the X-ray absorption part 2 (first X-ray absorption part 21) overlapped with the imaging part 11 of the pixel 20 in the plan view of the grid 1 is defined as X [μm] and the width of the imaging part 11 is defined as Y [μm], the value of X/Y is preferably in the range of 0.01 to 0.30 (see FIG. 2). In the case where the value of X/Y is within the above range, periodicity of the grid lines described above is surely maintained, and by reducing the difference between the pitch (W_{g}) of the X-ray absorption parts 2 in the grid 1 and the pitch (Wₚ) of the pixels 20, the grid lines can be made inconspicuous. Further, in the plan view of the grid 1, the area where the X-ray absorption part 2 and the imaging part 11 overlap is suppressed, and the X-rays can be made incident on the imaging part 11 more efficiently. As a result, a clearer high-quality X-ray image can be obtained, and this makes it possible to perform accurate X-ray imaging of the subject.

Further, the value of X/Y is more preferably in the range of 0.02 to 0.27, and even more preferably in the range of 0.04 to 0.25. As a result, the above-mentioned effects are remarkably exhibited.

Further, the pitch (W_{g}) of the X-ray absorption parts 2 of the grid 1 is preferably smaller than the pitch (Wₚ) of the pixels 20. This makes it possible to increase a grid density so that ability to remove scattered X-rays from the subject can be improved.

Further, in the grid-equipped X-ray detector 100 of the present embodiment, it is preferred that the X-ray absorption part 2 (the first X-ray absorption part 21) further overlaps with the non-imaging part 12 in the plan view of the grid 1. With this configuration, in the plan view of the grid 1, the area where the X-ray absorption part 2 overlaps with the imaging part 11 is reduced so that the X-rays passed through the subject are made incident on the imaging part 11 more efficiently. This makes it possible to perform more accurate X-ray imaging of the subject.

Further, in the grid-equipped X-ray detector 100 of the present embodiment, it is preferred that the X-ray transmission part 2 overlaps with the non-imaging part 12 in the plan view of the grid 1. With this configuration, the X-rays passed through the subject are made incident on the imaging part 11 more efficiently so that more accurate X-ray imaging of the subject can be performed.

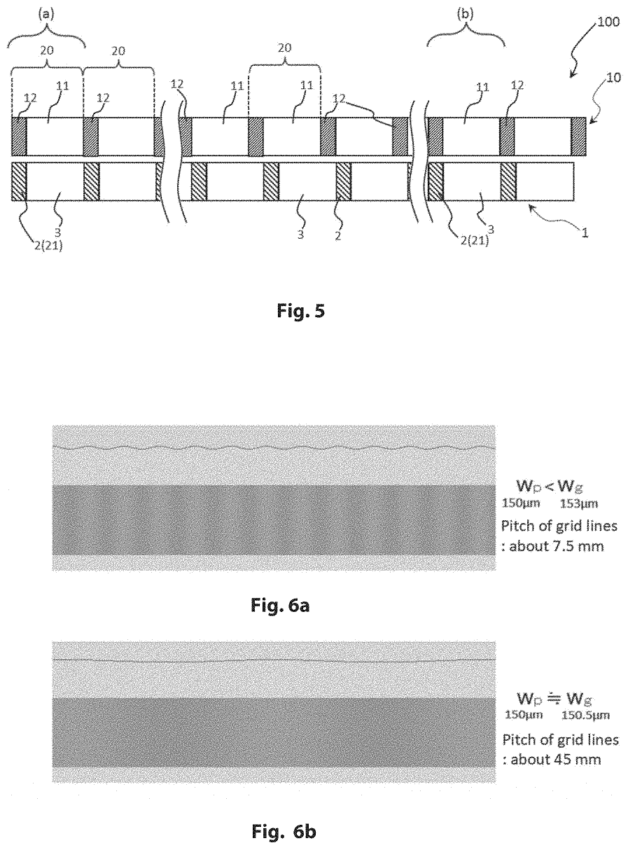

In the grid 1 of the grid-equipped X-ray detector 100 shown in FIG. 1, the width of the X-ray absorption part 2 is set to 20 μm and the width of the X-ray transmission part 3 is set to 130 μm. The grid-equipped X-ray detector 100 as shown in FIGS. 1 and 2 includes the region (a) having a portion where one left end of the X-ray absorption part 2 (the end opposite to the X-ray transmission part 3) and one left end of the non-imaging part 12 of the pixel 20 (the end opposite to the imaging part 11) are arranged so as to be offset from each other. However, the grid of the present invention (the grid-equipped X-ray detector of the present invention) is not limited to this, and for example, may have a configuration as shown in FIG. 5.

FIG. 5 is a cross-sectional view showing a state where a grid of another preferred embodiment of the present invention is attached to an X-ray detector.

In the grid 1 of the grid-equipped X-ray detector 100 shown in FIG. 5, the width of the X-ray absorption part 2 is set to 30 μm and the width of the X-ray transmission part 3 is set to 120 μm, and the grid 1 is attached to the X-ray detector 10 having the same pitch of the pixels as the X-ray detector 10 shown in FIG. 1. The grid-equipped X-ray detector 100 shown in FIG. 5 includes a region (a) having a portion where one left end of the X-ray absorption part 2 (the end opposite to the X-ray transmission part 3) overlaps with one left end of the non-imaging part 12 of the pixel 20 (the end opposite to the imaging part 11). Namely, in the grid-equipped X-ray detector 100 shown in FIG. 5, one end on the left side of the grid 1 and one end on the left side of the X-ray detector 10 are arranged so as to overlap to each other. Even with such a configuration, depending on a thickness of the adhesive that adheres the X-ray absorption part 2 and the X-ray transmission part 3, slight variation of the thickness of each member, and the like, at least a part of the X-ray absorption part 2 overlaps with the imaging part 11 of the X-ray detector 10 in the plan view of the grid 1, and thus the above-mentioned effect of the present invention is obtained.

In addition, the grid-equipped X-ray detector 100 as shown in FIG. 5 includes a region (a) having a portion where the one left end of the X-ray absorption part 2 overlaps with the one left end of the non-imaging part 12 of the pixel 20, and a region (b) having a portion where the one left end of the X-ray absorption part 2 overlaps with the one left end of the non-imaging part 12 of the pixel 20. When the X-ray absorption part 2 in each of the regions (a) and (b) is defined as the first X-ray absorption part 21, the first X-ray absorption parts 21 are present in the grid-equipped X-ray detector 100 as shown in FIG. 5 at a constant cycle due to the difference between the pitch (W_{g}) of the X-ray absorption parts 2 and the pitch (Wₚ) of the pixels 20. Namely, in the grid-equipped X-ray detector 100 shown in FIG. 5, the first X-ray absorption parts 21 are the plurality of X-ray absorption parts having the same overlapping state with the imaging parts 11 included in the grid 1 at a predetermined cycle. In this regard, in the grid-equipped X-ray detector 100 shown in FIG. 1, the first X-ray absorption parts 21 are not shown in the figure, but like the grid-equipped X-ray detector 100 shown in FIG. 5, the first X-ray absorption parts 21 are included therein.

The grid-equipped X-ray detector 100 is obtained by for example, providing the grid 1 adjacent to (or in close contact with) the surface on the subject side of the X-ray detector 10 using a fixing mechanism such as a fixed frame (not shown). Alternatively, the grid-equipped X-ray detector 100 is obtained by bonding (or adhering) the grid 1 to the surface of the X-ray detector 10 on the subject side via an adhesive substance (adhesive). At that time, it is preferable to align a center of the grid 1 with a center of the X-ray detector 10. However, as shown in FIG. 5, it is not always necessary to align one end of the X-ray absorption part 2 of the grid 1 with one end of the non-imaging part 12 of the pixel 20. This is because in the grid-equipped X-ray detector 100 of the present embodiment, the grid lines appearing in the obtained X-ray image are inconspicuous even if the grid 1 and the X-ray detector 10 are not strictly aligned so that a clear high-quality X-ray image can be obtained.

Further, the grid 1 attached to the X-ray detector 10 by the fixing mechanism or the adhesive substance can be easily attached to another X-ray detector by removing the grid 1 from the X-ray detector 10 by releasing the fixing mechanism or the like. Therefore, the grid 1 can be reused for a plurality of X-ray detectors having similar specifications.

While the grid and the grid-equipped X-ray detector of the present invention has been described based on the embodiment shown in the drawings hereinabove, the present invention shall not be limited thereto. Each structure constituting the grid and the grid-equipped X-ray detector may be substituted with an arbitrary structure having the same function as it. Further, arbitrary structures also may be added thereto.

### EXAMPLES

Next, an X-ray image taken by using the grid (grid-equipped X-ray detector) of the present invention will be described based on the specific examples shown below.

### Example 1

### Producing a Grid

First a lead foil having a thickness of 20 μm and an aluminum foil having a thickness of 126±1 μm were prepared.

Next, the lead foil and the aluminum foil were bonded with an epoxy adhesive, and then heat-dried. As a result, a bonded body in which the lead foil and the aluminum foil were bonded was obtained.

Next, the obtained bonded body was cut to obtain a plurality of strips having a length of 470 mm and a width of 3 mm. Then, the plurality of strips were laminated via the epoxy adhesive so that lead (X-ray absorption part) and aluminum (X-ray transmission part) were alternately arranged. After that, heat drying treatment was performed while pressurizing to prepare a grid having a length of 10 cm and a width of 10 cm. The pitch of the X-ray absorption parts of the obtained grid was 153 μm, the grid density was 65.3 lines/cm, and the grid ratio (lattice ratio) was 3:1, 4:1, 5:1.

### Producing a Grid-Equipped X-Ray Detector

Next, an X-ray detector as shown in FIG. 1 was prepared. The size of the X-ray detector was 35.04 cm in length×42.50 cm in width, and the pitch of the pixels was 150 μm (imaging part: 120 μm, non-imaging part: 30 μm).

Then, the produced grid was provided adjacent to the surface of the X-ray detector on the subject side in a state where the center of the grid and the center of the X-ray detector were aligned by using a fixing mechanism (not shown), so that a grid-equipped X-ray detector was obtained. In the grid-equipped X-ray detector thus obtained, the difference between the pitch (W_{g}) of the X-ray absorption parts of the grid and the pitch (Wₚ) of the pixels of the X-ray detector: the value of W_{g}−Wₚ was 3 μm.

### Example 2

### Producing a Grid

A grid was produced in the same manner as in Example 1 except that an aluminum foil having a thickness of 124±1 μm was used as the aluminum foil. The pitch of the X-ray absorption part of the obtained grid was 150.5 μm, the grid density was 66.4 lines/cm, and the grid ratio (lattice ratio) was 4:1.

### Producing a Grid-Equipped X-Ray Detector

An X-ray detector similar to that of Example 1 was prepared, and a grid-equipped X-ray detector was obtained in the same manner as in Example 1. The grid-equipped X-ray detector thus obtained had the value of W_{g}−Wₚ of 0.5 μm.

### Example 3

### Producing a Grid

A grid was produced in the same manner as in Example 1 except that an aluminum foil having a thickness of 124±1 μm was used as the aluminum foil. The pitch of the X-ray absorption part of the obtained grid was 149.5 μm, the grid density was 66.9 lines/cm, and the grid ratio (lattice ratio) was 4:1.

### Producing a Grid-Equipped X-Ray Detector

An X-ray detector similar to that of Example 1 was prepared, and a grid-equipped X-ray detector was obtained in the same manner as in Example 1. The grid-equipped X-ray detector thus obtained had the value of W_{g}−Wₚ of −0.5 μm.

### Comparative Example 1

### Producing a Grid

A grid was produced in the same manner as in Example 1 except that an aluminum foil having a thickness of 128±1 μm was used as the aluminum foil. The pitch of the X-ray absorption parts of the obtained grid was 155 μm, the grid density was 64.5 lines/cm, and the grid ratio (lattice ratio) was 3:1, 4:1, 5:1.

### Producing a Grid-Equipped X-Ray Detector

An X-ray detector similar to that of Example 1 was prepared, and a grid-equipped X-ray detector was obtained in the same manner as in Example 1. The grid-equipped X-ray detector thus obtained had the value of W_{g}−Wₚ of 5 μm.

### Evaluation of X-Ray Image (No Subject)

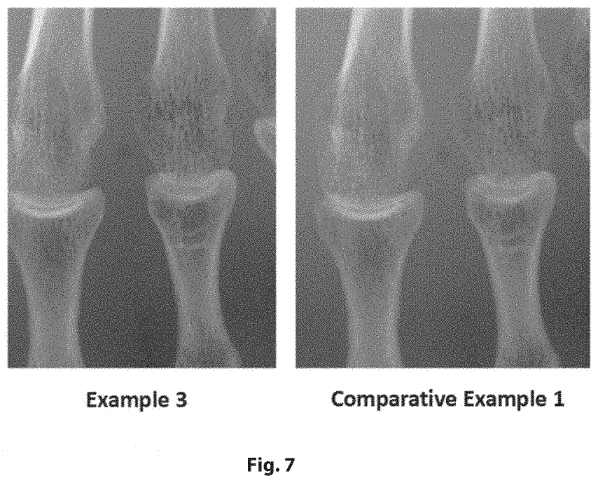

An X-ray tube (X-ray light source) and each of the grid-equipped X-ray detectors of Examples 1 and 2 were arranged so that a distance between an X-ray tube focus and the detector was 120 cm. Then, each grid-equipped X-ray detector was irradiated (exposed) with X-rays from an X-ray light source, and the grid lines appearing in the X-ray image taken by the X-ray detector were evaluated. X-ray irradiation conditions of the X-ray tube were tube voltage: 50 kv, tube current: 100 mA, and irradiation time: 50 msec. The results are shown in FIG. 6.

FIGS. 6(a) and (b) are diagrams showing the grid lines appearing in X-ray images obtained by directly irradiating the grid-equipped X-ray detectors of Examples 1 and 2 with X-rays.

As shown in FIGS. 6(a) and (b), when the difference between the pitch (W_{g}) of the X-ray absorption parts and the pitch (Wₚ) of the pixels is set to 3 μm or less, the grid lines appearing in the X-ray image have extremely low frequency. Specifically, the grid lines appearing in Example 1 have the pitch: about 7.5 mm and the frequency (spatial frequency): about 0.13 (cycles/mm). Similarly, the grid lines appearing in Example 2 have the pitch: about 45 mm and the frequency: about 0.02 (cycles/mm). Therefore, in the grid-equipped X-ray detector of Example 1, the cycle of the plurality of X-ray absorption parts having the same overlapping state as the imaging parts included in the grid was about 7.5 mm. Further, in the grid-equipped X-ray detector of Example 2, the cycle of the plurality of X-ray absorption parts having the same overlapping state as the imaging parts included in the grid was about 45 mm.

The grid lines shown in FIGS. 6(a) and 6(b) were more inconspicuous than the grid lines of FIGS. 3(a) to 3(c). In particular, in the grid-equipped X-ray detector of Example 2, the grid lines could not be visually recognized. Therefore, in the grid-equipped X-ray detectors of Examples 1 and 2, the influence of the grid lines appearing in the X-ray image could be reduced, and thus a clear high-quality X-ray image could be obtained.

Evaluation of X-Ray Image (with Subject)

X-ray imaging apparatuses in which the X-ray tube (X-ray light source) and each of the grid-equipped X-ray detectors of Example 3 and Comparative Example 1 were arranged so that the distance between the X-ray tube focus and the detector was 120 cm were prepared. An X-ray image was taken of a knee joint part of a human body with the X-ray imaging apparatus, and the obtained X-ray image was evaluated. X-ray irradiation conditions of the X-ray tube were tube voltage: 50 kv, tube current: 100 mA, and irradiation time: 50 msec. The results are shown in FIG. 7.

FIG. 7 is a diagram showing X-ray images obtained by imaging a subject (a knee joint part of a human body) using the grid-equipped X-ray detectors of Example 3 and Comparative Example 1.

As shown in FIG. 7, by using the grid-equipped X-ray detector of Example 3, it was possible to obtain a clear X-ray image in which no grid line was confirmed in the X-ray image. On the other hand, when the grid-equipped X-ray detector of Comparative Example 1 was used, a striped pattern showing the grid lines appeared in a vertical direction of the X-ray image, and a clear X-ray image could not be obtained.

### IND7TRIAL APPLICABILITY

According to the grid of the present invention, when the grid is viewed in a plan view in a state that the grid is provided adjacent to the surface of the X-ray detector, at least a part of the X-ray absorption part overlaps with the imaging part of the X-ray detector and an overlapping state of the X-ray absorption part and the imaging part transitions. Further, as a result of the transition of this overlapping state, the grid includes the plurality of X-ray absorption parts (the first X-ray absorption parts) having the same overlapping state with the corresponding imaging parts in a predetermined cycle, and the predetermined cycle is set within the range of 7.5 mm or more and 310 mm or less. By using the grid of the present invention, it is possible to obtain a clear high-quality X-ray image in which grid lines and grid artifacts are not visually recognized without exactly matching the pitch of the X-ray absorption parts of the grid with the pitch of the pixels of the X-ray detector and accurately aligning the grid with respect to the X-ray detector. As a result, this makes it possible to perform accurate X-ray imaging of a subject. Therefore, the present invention has industrial applicability.

1. A grid used to be with an X-ray detector to take an X-ray image of a subject, wherein the X-ray detector is configured by two-dimensionally arranging a plurality of pixels each including an imaging part for detecting incident X-rays and a non-imaging part provided adjacent to the imaging part, the grid comprising:
a plurality of X-ray absorption parts that absorb the X-rays; and
a plurality of X-ray transmission parts that transmit the X-rays,
wherein the grid is configured by alternately arranging the plurality of X-ray absorption parts and the plurality of X-ray transmission parts and adapted to be externally provided adjacent to a surface of the X-ray detector,
wherein when the grid is viewed in a plan view in a state that the grid is provided adjacent to the surface of the X-ray detector, at least a part of the X-ray absorption part overlaps with the imaging part and an overlapping state of the X-ray absorption part and the imaging part transitions,
wherein the plurality of X-ray absorption parts include first X-ray absorption parts having one end which overlaps with one end of the non-imaging parts, and
wherein the first X-ray absorption parts are included in the grid at a predetermined cycle and the predetermined cycle is 7.5 mm or more and 310 mm or less.

2. The grid as claimed in claim 1, wherein the X-ray absorption part further overlaps with the non-imaging part.

3. The grid as claimed in claim 1, wherein when a width at which the at least part of the X-ray absorption part overlaps with the imaging part is defined as X [μm] and a width of the imaging part is defined as Y [μm], a value of X/Y is in a range of 0.01 to 0.30.

4. The grid as claimed in claim 1, wherein at least a part of the X-ray transmission part overlaps with the non-imaging part.

5. The grid as claimed in claim 1, wherein a pitch of the plurality of X-ray absorption parts is smaller than a pitch of the plurality of pixels.

6. The grid as claimed in claim 1, wherein the X-ray transmission part is made of a paper fibrous base material impregnated with an epoxy resin.

7. A grid-equipped X-ray detector comprising:
an X-ray detector configured by two-dimensionally arranging a plurality of pixels each including an imaging part for detecting incident X-rays and a non-imaging part provided adjacent to the imaging part; and
a grid having a plurality of X-ray absorption parts that absorb the X-rays and a plurality of X-ray transmission parts that transmit the X-rays, the grid configured by alternately arranging the plurality of X-ray absorption parts and the plurality of X-ray transmission parts and adapted to be externally provided adjacent to a surface of the X-ray detector,
wherein when the grid is viewed in a plan view in a state that the grid is provided adjacent to the surface of the X-ray detector, at least a part of the X-ray absorption part overlaps with the imaging part of the X-ray detector and an overlapping state of the X-ray absorption part and the imaging part transitions,
wherein the plurality of X-ray absorption parts include first X-ray absorption parts having one end which overlaps with one end of the non-imaging parts, and
wherein the first X-ray absorption parts are included in the grid at a predetermined cycle and the predetermined cycle is 7.5 mm or more and 310 mm or less.
